(19) United States
(12) Patent Application Publication
(10) Pub. No.: US 2024/0392229 A1
(43) Pub. Date: Nov. 28, 2024

(54) DATA OPTIMIZATION METHOD AND SYSTEM FOR FOOD FERMENTATION PROCESS

(71) Applicants: JIMEI UNIVERSITY, Xiamen (CN); JIANGSU UNIVERSITY, Zhenjiang (CN)

(72) Inventors: Quansheng CHEN, Xiamen (CN); Songguang ZHAO, Zhenjiang (CN); Hao LIN, Zhenjiang (CN); Zhen WANG, Zhenjiang (CN); Tianhui JIAO, Xiamen (CN); Qin OUYANG, Zhenjiang (CN); Huanhuan LI, Zhenjiang (CN)

(21) Appl. No.: 18/791,357

(22) Filed: Jul. 31, 2024

(30) Foreign Application Priority Data
Jun. 12, 2024 (CN) 202410750353.7

(51) Int. Cl. C12M 1/36; C12M 1/34

(52) U.S. Cl. CPC C12M 41/48; C12M 41/26; C12M 41/34; C12M 41/36

(57) ABSTRACT

A data optimization method for a food fermentation process is provided. Process data of the food fermentation process is acquired, and a multi-scale cross-correlation feature filter (MCFF) is constructed. Feature data corresponding to the process data is extracted in real time based on the MCFF, and processed. A data prediction model corresponding to the feature data is created through a machine learning method, and based on the data prediction model and an optimization algorithm, predicted optimization control data corresponding to the food fermentation process is generated in real time. A data optimization system is further provided.

### CROSS-REFERENCE TO RELATED APPLICATIONS

This application claims the benefit of priority from Chinese Patent Application No. 202410750353.7, filed on Jun. 12, 2024. The content of the aforementioned application, including any intervening amendments thereto, is incorporated herein by reference in its entirety.

### TECHNICAL FIELD

This application relates to food fermentation, and more particularly to a data optimization method and system for a food fermentation process.

### BACKGROUND

Food fermentation, as an important traditional food processing technology, has been widely used in the food industry. The food fermentation technology has been constantly optimized to satisfy higher and higher requirements for food safety, nutrition and quality. Real-time control of the food fermentation process is an effective measure to ensure product consistency and stability. The food fermentation process is characterized by strong nonlinearity, obvious time-delay characteristic, and strong coupling and dynamic characteristics, and moreover, the food fermentation process is complex, and susceptible to many factors, thereby making the modeling, optimization and control of the fermentation process extremely difficult and complicated.

The fermentation process model is generally designed to describe the mapping relationship between the input parameters and output parameters in the fermentation process, and the predicted output of the fermentation process model is used to achieve optimization control. The fermentation process model mainly includes a fermentation mechanism-based white-box model and a data-driven black-box model. The white-box model usually requires in-depth study of the fermentation mechanism, but because the fermentation process is relatively complex, and susceptible to many uncertain factors, the establishment of the white-box model has become particularly difficult. By comparison, the process data is more accessible in the practical production, so the black-box model, which only considers input and output variables and does not involve the fermentation mechanism, is preferred for the modelling of complex systems.

In addition, there is a coupling effect between individual parameters in the fermentation process, and the regulation of a single parameter may cause a chain reaction of other parameters, resulting in an uncontrollable fermentation process.

Therefore, in view of the above technical deficiencies, there is an urgent need to design and develop a data optimization method and system suitable for the food fermentation process.

### SUMMARY

In view of the above-mentioned deficiencies and difficulties in prior art, this application provides a data optimization method and system of a food fermentation process, in which the high-level transformation of data features and deep mining of process data are achieved to establish a reliable process model, and based on the process model and an optimization algorithm, an optimization control system is constructed, thereby solving the problem of mutual coupling between individual process parameters.

In a first aspect, this application provides a data optimization method of a food fermentation process, comprising:
(a) acquiring process data of the food fermentation process; wherein the process data comprises environmental parameter data and fermentation system parameter data;
(b) constructing a multi-scale cross-correlation feature filter (MCFF); extracting feature data corresponding to the process data in real time based on the MCFF; and processing the feature data by collaborative hybrid; and
(c) creating a data prediction model corresponding to the feature data through a machine learning method; and based on the data prediction model in combination with an optimization algorithm, generating predicted optimization control data corresponding to the food fermentation process in real time; wherein the machine learning method comprises a linear method and a non-linear method; and the optimization algorithm is a swarm intelligence algorithm or a meta-heuristic algorithm.

In an embodiment, the step (a) further comprises:
(a1) constructing a process data set corresponding to the process data, and processing process data in the process data set by interval scaling (IS); and
(a2) coupling the process data at a current moment with target value data at a next moment.

In an embodiment, the step (a1) further comprises:
splitting the process data set into a training set and a test set according to a preset ratio through an input-output cooperative distance classification (IOCDC) method, wherein a cooperative distance calculation formula is expressed as:

$${d}_{xy} ( i , j ) = \frac{{d}_{x} ( i , j )}{{max}_{i , j ∈ ( 1 , z )} ( {d}_{x} ( i , j ) )} + \frac{{d}_{y} ( i , j )}{{max}_{i , j ∈ ( 1 , z )} ( {d}_{y} ( i , j ) )} ; \qquad ( 1 )$$

wherein i, j∈[1, z], z is the number of samples; dₓ(i, j) is an input-based inter-sample distance; and d_{y}(i, j) is an output-based inter-sample distance.

In an embodiment, the step (a1) further comprises:
processing the process data in the process data set in real time by IS, wherein an IS function is expressed as follows:

$${X}_{i} = ( {X}_{oi} - {X}_{oi , min} ) / ( {X}_{oi , max} - {X}_{oi , min} ) ; \qquad ( 2 )$$

wherein Xₒᵢ is real-time feature data before the IS; and X_{oi,max} and X_{oi,min} represent maximum value and minimum value of individual calculation dimensions, respectively.

In an embodiment, the step (b) further comprises:
adjusting a size of each of at least one filter module to construct the MCFF in real time, wherein the MCFF comprises the at least one filter module and at least one splicing module; the number of the at least one filter module is adjustable; each of the at least one filter module comprises a filtering sub-module, a batch normalization sub-module, an activation sub-module, and a pooling sub-module; the at least one filter module has the same size; and the at least one splicing module is configured for integrating an output of the at least one filter module; and
processing the feature data in real time by using a corresponding one of the at least one filter module through steps of:
extracting, by the filtering sub-module, features of an input parameter; processing, by the batch normalization sub-module, filtered data to reduce an overfitting probability; performing, by the activation sub-module, a non-linear mapping of normalization data; and reducing, by the pooling sub-module, a dimensionality of mapped data.

In an embodiment, the step (c) further comprises:
generating and acquiring indicator regulation data corresponding to the food fermentation process in real time;
establishing an indicator regulation process prediction model corresponding to the indicator regulation data, and generating optimization algorithm parameter data corresponding to the indicator regulation process prediction model; and
encoding the indicator regulation data; and optimizing and regulating the food fermentation process in real time according to the optimization algorithm parameter data in combination with sampling frequency data or sampling interval data of the process data; wherein the indicator regulation data is encoded by floating-point encoding and binary encoding.

In a second aspect, this application provides a data optimization system of a food fermentation process, comprising:
a data acquisition unit;
a construction and extraction unit; and
a creation and generation unit;
wherein the data acquisition unit is configured to acquire process data of the food fermentation process; wherein the process data comprises environmental parameter data and fermentation system parameter data;
the construction and extraction unit is configured to construct a multi-scale cross-correlation feature filter (MCFF), extract feature data corresponding to the process data in real time based on the MCFF, and process the feature data by collaborative hybrid; and
the creation and generation unit is configured to create a data prediction model corresponding to the feature data through a machine learning method, and generate predicted optimization control data corresponding to the food fermentation process in real time based on the data prediction model in combination with an optimization algorithm; wherein the machine learning method comprises a linear method and a non-linear method; and the optimization algorithm is a swarm intelligence algorithm or a meta-heuristic algorithm.

In an embodiment, the data acquisition unit comprises a first data processing module and a second data processing module; the first data processing module is configured for constructing a process data set corresponding to the process data and processing the process data in the process data set by interval scaling (IS); and the second data processing module is configured for coupling the process data at a current moment with target value data at a next moment;
the construction and extraction unit comprises a first construction module and a third data processing module; the first construction module is configured for adjusting a size of each of at least one filter module to construct the MCFF in real time, wherein the MCFF comprises the at least one filter module and at least one splicing module, and the number of the at least one filter module is adjustable; each of the at least one filter module comprises a filtering sub-module, a batch normalization sub-module, an activation sub-module, and a pooling sub-module; the at least one filter module has the same size; and the at least one splicing module is configured for integrating an output of the at least one filter module;
the third data processing module is configured for processing the feature data in real time by using a corresponding one of the at least one filter module through steps of:
extracting features of an input parameter by the filtering sub-module; processing filtered data to reduce an overfitting probability by the batch normalization sub-module; performing a non-linear mapping of normalization data by the activation sub-module; and reducing a dimensionality of mapped data by the pooling sub-module;
the creation and generation unit comprises a data acquisition module, a data generation module and an optimization and regulation module; the data acquisition module is configured for generating and acquiring indicator regulation data corresponding to the food fermentation process in real time;
the data generation module is configured for establishing an indicator regulation process prediction model corresponding to the indicator regulation data, and generating optimization algorithm parameter data corresponding to the indicator regulation process prediction model; and
the optimization and regulation module is configured for encoding the indicator regulation data; and optimizing and regulating the food fermentation process in real time according to the optimization algorithm parameter data in combination with sampling frequency data or sampling interval data of the process data; wherein the indicator regulation data is encoded by floating-point encoding and binary encoding.

In an embodiment, the first data processing module comprises a dataset splitting module configured for splitting the process data set into a training set and a test set through an input-output cooperative distance classification (IOCDC) method.

In an embodiment, the first data processing module comprises an IS processing module configured to process the process data in real time through IS.

This application has the following beneficial effects.

A data optimization method provided in this application acquires process data of the food fermentation process. The process data includes environmental parameter data and fermentation system parameter data. The method constructs the MCFF, extracts feature data corresponding to the process data in real time based on the MCFF, and processes the feature data by collaborative hybrid. The method creates a data prediction model corresponding to the feature data through a machine learning method, and based on the data prediction model in combination with an optimization algorithm, generates predicted optimization control data corresponding to the food fermentation process in real time. The machine learning method includes a linear method and a non-linear method. The optimization algorithm is a swarm intelligence algorithm or a meta-heuristic algorithm. The data optimization method and the data optimization system corresponding to the method realize a high-level transformation of the features and complete process data deep mining to establish a reliable process model. Based on the process model, the optimization control system is built in combination with the optimization algorithm, which solves the problem of mutual coupling of process parameters.

In view of the challenges of modelling and optimization control in the food fermentation process, and considering the high-level feature extraction of process data, this application designs a data-driven and multi-scale feature extraction-based method for modelling and optimization control in the food fermentation process. The MCFF feature extraction tool is designed to achieve multi-scale feature extraction of process data through filtering operations, and collaboratively mix feature data at different scales, thereby achieving high-level transformation of features and completing the deep mining of process data to establish a reliable process model. The optimization control system is built based on the process model and the optimization algorithm, thereby solving the difficult problem of the process parameters coupling with each other. The accuracy of the prediction model and the effectiveness of the optimization control method are verified by taking the fermentation process of kombucha as an example.

### BRIEF DESCRIPTION OF THE DRAWINGS

In order to illustrate the technical solutions in the embodiments of the present disclosure more clearly, the drawings required in the description of the embodiments will be briefly described below. Obviously, presented in the drawings are merely some embodiments of the present disclosure, which are not intended to limit the disclosure. For those skilled in the art, other drawings may also be obtained according to the drawings provided herein without paying creative efforts.

FIG. 1 is a schematic diagram of construction of process data in a data optimization method according to one embodiment of the present disclosure;

FIG. 2 is a schematic diagram of a single-scale filter of the data optimization method according to one embodiment of the present disclosure;

FIG. 3 is a schematic diagram of multi-scale feature data collaborative hybrid and collaborative hybrid data training in the data optimization method according to one embodiment of the present disclosure;

FIG. 4 is a schematic diagram of multi-scale feature convolutional collaborative hybrid in the data optimization method according to one embodiment of the present disclosure;

FIG. 5 schematically shows a structure of an optimization control system in the data optimization method according to one embodiment of the present disclosure;

FIG. 6A shows validation results of a C source concentration prediction model in the data optimization method according to one embodiment of the present disclosure;

FIG. 6B shows validation results of a bacterial concentration prediction model in the data optimization method according to one embodiment of the present disclosure;

FIG. 7A shows simulation results of optimization control of C source concentration in the data optimization method according to one embodiment of the present disclosure;

FIG. 7B shows pH regulation under the C-source concentration optimization control in the data optimization method according to one embodiment of the present disclosure;

FIG. 7C shows DO regulation under the C source concentration optimization control in the data optimization method according to one embodiment of the present disclosure;

FIG. 7D shows simulation results of optimization control of bacterial concentration in the data optimization method according to one embodiment of the present disclosure;

FIG. 7E shows pH regulation under the bacterial concentration optimization control in the data optimization method according to one embodiment of the present disclosure;

FIG. 7F shows DO regulation under the bacterial concentration optimization control in the data optimization method according to one embodiment of the present disclosure;

FIG. 8A shows bacterial concentration in a segmented optimization control process of the data optimization method according to one embodiment of the present disclosure;

FIG. 8B shows C source concentration in the segmented optimization control process of the data optimization method according to one embodiment of the present disclosure;

FIG. 8C shows DO regulation (dissolved oxygen is needed to be regulated throughout the fermentation process) in the segmented optimization control process of the data optimization method according to one embodiment of the present disclosure;

FIG. 8D shows pH regulation in the segmented optimization control process of the data optimization method according to one embodiment of the present disclosure;

FIG. 9 is a flow chart of the data optimization method according to one embodiment of the present disclosure; and

FIG. 10 schematically shows a structure of a data optimization system for a food fermentation process.

The objectives, functional features and advantages of the present disclose will be further illustrated below in combination with the embodiments and the accompanying drawings.

### DETAILED DESCRIPTION OF EMBODIMENTS

The disclosure will be described in detail below in combination with the drawings and the embodiments to make the technical solutions, objects and advantages of the disclosure clearer.

The disclosure can also be implemented or applied in other ways. For those skilled in the art, other embodiments obtained based on these embodiments without paying creative efforts should fall within the scope of the disclosure.

As used herein, it should be noted that terms, such as “up”, “down”, “left”, “right”, “front”, or “rear”, are only used to describe the relative positional relationship or movement between individual components in a particular attitude (as shown in the accompanying drawings), and if the particular attitude changes, the orientation indications will also change accordingly. Therefore, these terms should not be understood as a limitation of the present disclosure.

In addition, the terms “first” and “second” are merely descriptive, and cannot be understood as indicating or implying relative importance or the number of the technical features. As a result, a feature defined with “first” or “second” may include at least one such feature either explicitly or implicitly. It should be noted that embodiments of the present disclosure and the features therein may be combined with each other in the case of no contradiction.

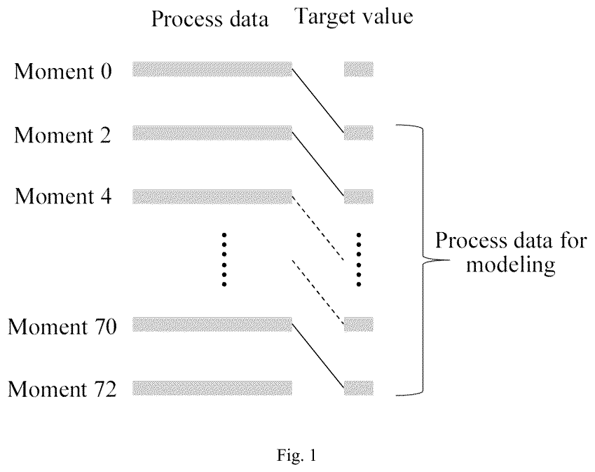

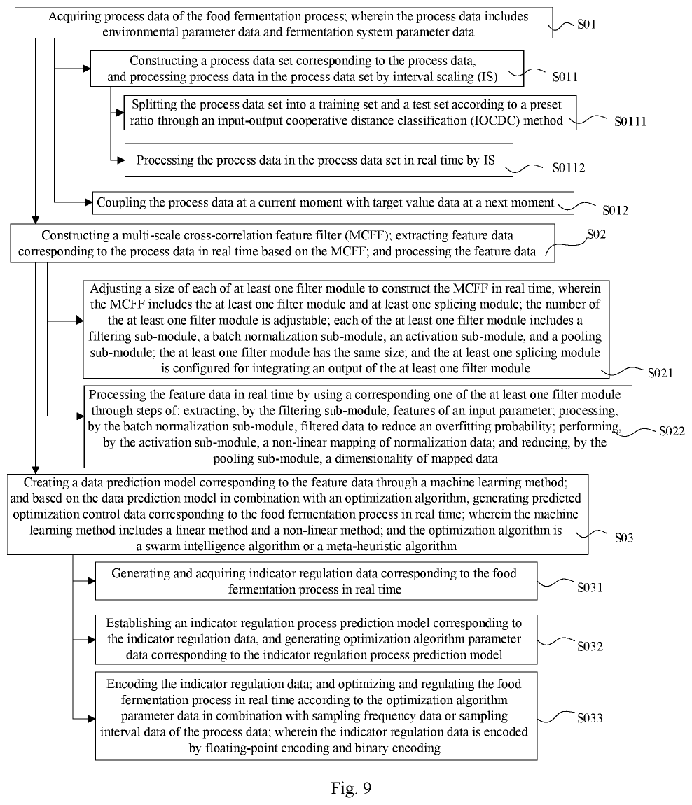

The disclosure will be further described in detail below in conjunction with the accompanying drawings. As shown in FIGS. 1-9, the disclosure provides a data optimization method applicable to a food fermentation process.
(S01) Process data corresponding to the food fermentation process is acquired, which includes environmental parameter data and fermentation system parameter data.
(S02) A multi-scale cross-correlation feature filter (MCFF) is constructed. Based on the MCFF, feature data corresponding to the process data is extracted in real time, and the extracted feature data is processed by collaborative hybrid.
(S03) A data prediction model corresponding to the feature data is created through a machine learning method. Based on the data prediction model in combination with a corresponding optimization algorithm, predicted optimization control data corresponding to the food fermentation process is generated in real time. In an embodiment, the machine learning method includes a linear method and a non-linear method. The optimization algorithm is a swarm intelligence algorithm or a meta-heuristic algorithm.

The step (S01) further includes the following steps.
(S011) A process data set corresponding to the process data is constructed, and process data in the process data set is processed by interval scaling (IS).
(S012) The process data at a current moment is coupled with target value data at a next moment.

The step (S011) further includes the following steps.
(S0111) The process data set is split into a training set and a test set according to a preset ratio through an input-output cooperative distance classification (IOCDC) method. The preset ratio of the training set to the test set is 7:3. A cooperative distance calculation formula is expressed as:

$${d}_{xy} ( i , j ) = \frac{{d}_{x} ( i , j )}{{max}_{i , j ∈ ( 1 , z )} ( {d}_{x} ( i , j ) )} + \frac{{d}_{y} ( i , j )}{{max}_{i , j ∈ ( 1 , z )} ( {d}_{y} ( i , j ) )} . \qquad ( 1 )$$

In the above formula, i, j∈[1, z], z is the number of samples; dₓ(i, j) is an input-based inter-sample distance; and d_{y}(i, j) is an output-based inter-sample distance.

The step (S011) further includes the following steps.
(S0112) The process data is processed in real time through IS, where an IS function is expressed as follows:

$${X}_{i} = ( {X}_{oi} - {X}_{oi , min} ) / ( {X}_{oi , max} - {X}_{oi , min} ) . \qquad ( 2 )$$

In the above formula, Xₒᵢ is real-time feature data before the IS; and X_{oi,max} and X_{oi,min} represent maximum value and minimum value of individual calculation dimensions, respectively.

In an embodiment, the data processing sequence is acquisition of the data, construction of the process data set (coupling process data with target values), IS processing of the corresponding data, and data set split ting by IOCDC method successively.

The step (S02) further includes the following steps.
(S021) A size of each of at least one filter module is adjusted to construct MCFFs in real time. The MCFF includes the at least one filter module and at least one splicing module. The number of the at least one filter module is adjustable. Each of the at least one filter module includes a filtering sub-module, a batch normalization sub-module, an activation sub-module, and a pooling sub-module. The at least one filter module has the same size. The at least one splicing module is configured for integrating an output of at least one filter module.
(S022) The feature data is processed in real time by using a corresponding one of at least one filter module through steps of: extracting features of input parameters by the filtering sub-module; processing filtered data to reduce an overfitting probability by the batch normalization sub-module; performing a non-linear mapping of normalization data by the activation sub-module; and reducing a dimensionality of mapped data by the pooling sub-module.

The step (S03) further includes the following steps.
(S031) Indicator regulation data corresponding to the food fermentation process is generated and acquired in real time.
(S032) An indicator regulation process prediction model corresponding to the indicator regulation data is established, and optimization algorithm parameter data corresponding to the indicator regulation process prediction model is generated.
(S033) The indicator regulation data is encoded; and according to the optimization algorithm parameter data in combination with sampling frequency data or sampling interval data of the process data, the food fermentation process is optimized and regulated in real time. The indicator regulation data is encoded by floating-point encoding and binary encoding.

In an embodiment, a modelling, optimization and control method for the food fermentation process based on data-driven and multi-scale cross-correlation feature filter provided herein includes two major parts of fermentation process modelling and optimization control. The established model is a prediction model, where based on process data at time t, the concentration of the regulation indicators at time t+a is predicted.

In an embodiment, the prediction model is established through the following steps: (S1) collecting process data of key indicators in the fermentation process; (S2) constructing a process data set, and coupling the process data at the current moment with the target value at the next moment; (S3) normalizing the constructed process data; (S4) constructing an MCFF based on the data in (S3) and extracting the features of the process data; (S5) collaboratively mixing the extracted features based on the data in (S4); (S6) putting the co-mixed features into a machine learning method to build a prediction model for prediction.

In step (S3), differences between different dimensions are eliminated by IS.

In step (S4), the multi-scale filter is constructed. The multi-scale filter includes the at least one filter module and one splicing module. The number of the at least one filter module is adjustable. Each filter module includes the filtering sub-module, the batch normalization sub-module, the activation sub-module, and the pooling sub-module. The at least one filter module has the same size. The multi-scale filters are constructed by adjusting sizes of the filter modules. The filtering sub-modules are used to extract local features of the input parameters. The batch normalization sub-module is used to process the filtered data to reduce the overfitting possibility. The activation sub-module is used to perform non-linear mapping of normalization data. The pooling sub-module is used to reduce the dimensionality of the feature data. The splicing module is used to integrate the outputs of the multi-scale filters.

In step (S5), the features extracted from the multi-scale filters are collaboratively mixed. The features extracted from the multi-scale filters have the same size of feature maps. The collaborative hybrid methods include average collaborative hybrid, attention weighted average collaborative hybrid, and convolutional collaborative hybrid. The feature collaborative hybrid realizes the integration and dimensionality reduction of the feature data from the multi-scale filters.

In step (S6), the collaborative hybrid data is used as an input to the machine learning method for process model training. The machine learning method mainly includes the linear method and the non-linear method.

The process prediction model is used instead of expert experience, and the hybrid optimization algorithm solves the optimal parameter combinations to regulate the fermentation process in real time, which specifically include the following steps: (S10) determination of the regulation indicators; (S20) establishment of the process model of the regulation indicators; (S30) parameter setting of the optimization algorithm; (S40) individual coding; (S50) building of an fitness function; and (S60) determination of a sampling frequency or interval.

In step (S10), the regulation indicators are the indicators with production guidance significance. In step (S20), the process model is to establish the prediction model based on the above method to obtain the prediction value of the regulation indicators, which is used to observe the change trend of the regulation indicators. In step (S30), the optimization algorithm is the meta-heuristic algorithm, and parameters to be set mainly include the number of individuals, the maximum number of iterations, the range of individual gene changes, and the length of the individuals. In step (S40), the individual encoding method includes floating-point encoding and binary encoding. In step (S50), the fitness function provides a reference fitness value for the optimization algorithm and guides the optimization algorithm to converge in the target direction. The main functions of the fitness function include, but are not limited to, prediction of the model, processing of the prediction results, and other fermentation constraints. The prediction of the model refers to the model prediction of the regulation parameters. The processing of the prediction results refers to processing the prediction results of the regulation target, including derivation, summation, difference, or averaging. The other fermentation constraints refer to consider whether the change of other parameters is within a reasonable range when performing the optimization control of the regulation target: if it is out of the range, the obtained result may be a pseudo-result, which is detrimental to the fermentation process. In step (S60), the sampling frequency is reasonably designed according to the fermentation cycle, and the sampling frequency should not be too high or too low. If the sampling frequency is too high, the number of regulation times is high, which will increase the cost of fermentation. If the sampling frequency is too low, the number of regulation times is low, which will miss the key time point for adjusting the fermentation process, resulting in a decrease in yield.

Since the food fermentation process has strong nonlinearity, time lag, and dynamics, the fermentation process mechanism is complex and susceptible to uncertainties, which brings difficulties in the establishment of process models and the implementation of optimization control. It is necessary to establish a reliable process model to achieve the optimization control of the fermentation process to ensure the high quality and stable operation of the fermentation process.

The establishment of the stable and reliable process model is a prerequisite for optimization control. In practice, it is relatively easy to obtain process data, so black-box models, which only need to consider input and output variables but not the fermentation process mechanism, are widely focused. Black-box models are mostly built using classical machine learning methods. In recent years, feature engineering, which is crucial for improving model performance, has received increasing attention. By designing filters at different scales, features from the original data hierarchy can be learnt. Not only basic features can be extracted, but also these features can be collaboratively mixed to obtain more abstract and complex advanced features.

In the optimization control strategy, the key parameters that need to be adjusted are integrated into a one-dimensional vector to achieve simultaneous optimization of multiple variables, thereby solving the problem of parameter coupling. The fermentation process model and the optimization algorithm are used together to form the optimization control system to solve the optimal parameter combination. Commonly used optimization methods include Genetic Algorithm (GA), Particle Swarm Optimization (PSO), Grey Wolf Optimization (GWO), Whale Optimization Algorithm (WOA), simulated annealing (SA) and other meta-heuristic algorithms.

In other words, an object of the disclosure is to provide a data-driven and multi-scale feature extraction-based method for modelling, optimizing and controlling a food fermentation process and to applying method to the fermentation process of kombucha.

In order to achieve the above object, the disclosure designs a multi-scale cross-correlation feature filter (MCFF) for extracting features of process data in order to build a fermentation process prediction model, and build an optimization control system based on the fermentation process prediction model using an optimization algorithm.

The described method firstly uses MCFF to extract features from the input parameters of the fermentation process; secondly, the extracted features are performed with collaborative hybrid and dimension reduction; then the collaborative hybrid features are used as inputs to the classical machine learning method to train the process model; subsequently, the predicted values of the process model are used as the basis of the optimization control; and finally, the optimal combination of fermentation parameters is solved by using the optimization search method.

The process model is a prediction model, where based on the process data at moment t, the regulation indicators at moment t+a is predicted, and a is the sampling time interval.

Optionally, the process model is established through the following steps. Step 1, key process data in the fermentation process is collected. The key process data is divided into environmental parameters and fermentation system key parameters. The environmental parameters mainly include temperature, pH, dissolved oxygen, and stirring rate. The fermentation system key parameters mainly include bacterial concentration, nutrients, products. Step 2, the features of process data are extracted using the MCFF framework. The MCFF framework includes different scales of MCFFs to extract process data features from different perspectives. Each MCFF includes filter modules and one splicing module. The number of the filter modules is adjustable. Each filter module includes the filtering sub-module, the batch normalization sub-module, the activation sub-module, and the pooling sub-module. The filter modules have the same size. Different scales of MCFFs are constructed by adjusting the size of each filter module. The filtering sub-module is used to extract the features of the input parameters. The batch normalization sub-module is used to process the filtered data to reduce the overfitting possibility. The activation sub-module is used to achieve non-linear mapping. The pooling sub-module is used to reduce a dimensionality of mapped data. The splicing module is used for integrating the output of the MCFF. In step 3, the features extracted from the different scales of the MCFFs are performed with collaborative hybrid. The features extracted from the different scales of the MCFFs have the same size of the feature maps. The collaborative hybrid methods include average collaborative hybrid, attention weighted average collaborative hybrid, and convolutional collaborative hybrid. The feature collaborative hybrid realizes the integration and dimensionality reduction of the feature data from different scales of MCFFs. In step 4, the collaborative hybrid data is used as an input to the machine learning method for process model training. The machine learning method mainly includes the linear method and the non-linear method.

Optionally, the construction of the optimization control system includes the following steps. Step 1, determination of regulation indicators: the regulation indicators refer to the indicators with production guidance significance. Step 2, establishment of the process model of the regulation indicators: the prediction model is established based on the above method to obtain the prediction value of the regulation indicators, which is used to observe the change trend of the regulation indicators. Step 3, parameter setting of the optimization algorithm: the optimization algorithm is the meta-heuristic algorithm, and parameters to be set mainly include the number of individuals, the maximum number of iterations, the range of individual gene changes, and the length of the individuals. Step 4, individual coding: the individual encoding method includes floating-point encoding and binary encoding. Step 5, the fitness function: the fitness function provides reference fitness values for the optimization algorithm and guides the optimization algorithm to converge in the target direction; the main functions of the fitness function include, but are not limited to, prediction of the model, processing of the prediction results, and other fermentation constraints; the prediction of the model refers to the model prediction of the regulation parameters; the processing of the prediction results refers to processing the prediction results of the regulation target, including derivation, summation, difference, or averaging; the other fermentation constraints refer to consider whether the change of other parameters is within a reasonable range when performing the optimization control of the regulation target: if it is out of the range, the obtained result may be the pseudo-result, which is detrimental to the fermentation process. Step 6, determination of the sampling frequency or interval: the sampling frequency is reasonably designed according to the fermentation cycle, and the sampling frequency should not be too high or too low; if the sampling frequency is too high, the number of regulation times is high, which will increase the cost of fermentation; if the sampling frequency is too low, the number of regulation times is low, which will miss the key time point for adjusting the fermentation process, resulting in a decrease in yield.

The disclosure also provides a method for modelling and optimally controlling a kombucha fermentation process. The disclosure adopts the above method for modelling and optimally controlling the kombucha fermentation process. The key process data includes fermentation time, bacterial concentration, substrate concentration, product concentration, dissolved oxygen, pH value, and theanine. The regulation indicators refer to bacterial concentration and substrate concentration.

Optionally, the process model parameters include filter size, number of filters, number of MCFFs, learning rate, and data collaborative hybrid. The optimization control system parameters include number of individuals in the optimization method, maximum number of iterations, length of individuals, range of individual gene changes, individual coding method, and building of fitness function. The disclosure also provides the application of the above method in the field of fermentation.

In view of the challenges of modelling and optimization control in the food fermentation process, and considering the high-level feature extraction of process data, this application designs a data-driven and multi-scale feature extraction-based approach for modelling and optimization control in the food fermentation process. The MCFF feature extraction tool is designed to achieve multi-scale feature extraction of process data through filtering operations, and collaboratively mix feature data at different scales, thereby achieving high-level transformation of features and completing the deep mining of process data to establish a reliable process model. The optimization control system is built based on the process model and the optimization algorithm, thereby solving the difficult problem of the process parameters coupling with each other. The accuracy of the prediction model and the effectiveness of the optimization control method are verified by taking the fermentation process of kombucha as an example.

### Embodiment Modelling and Optimization Control in the Fermentation Process of Kombucha

Kombucha is a fermented tea beverage, which is made by fermenting tea-sugar water with various probiotics such as yeasts, and is popular among consumers for its unique flavor and health benefits.

The fermentation process of kombucha is a non-linear, time-delay and dynamic process. The experiments were carried out in a 5 L bioreactor with temperature of 30° C. and stirring speed of 150 rpm. The variable environmental parameters were Dissolved Oxygen (DO) and pH, which were also used as variables for optimization control. The fermentation cycle was 72 h, the sampling interval was 2 h, and 37 samples were collected from each batch. Seven indicators, including the current fermentation moment, bacterial concentration, C source, total acid, DO, pH, and theanine (a flavor substance in the tea), were selected as process variables. A total of five batches of data under the normal production conditions were collected, of which four batches were used as training samples and the remaining one batch was used as test samples to test the validity of the process model. The specific steps were as follows.

### Step 1 Determination of Regulation Objectives

The fermentation process of kombucha followed the law of food fermentation process, which was divided into four stages: adaptation, logarithmic growth, stabilization and decline. Optionally, this study mainly regulated the concentration of the bacteria in the logarithmic growth period and the consumption of C source in the stabilization period, in order to improve the fermentation efficiency and reduce the cost of fermentation. Therefore, the prediction models of the concentration of the bacteria and the C source were established.

### Step 2 Construction of Process Data

Optionally, the process data of the first 36 samples of each batch was used to couple with the target value at the next moment. As shown in FIG. 1, 36 sets of process data were obtained for each batch, and a total of 144 sets of process data were obtained for the four batches. 6 indicators in the process variables were used to construct the process data, and one indicator was used as the target value.

### Step 3 Pre-Processing of Process Data

Due to the different scale and unit of process data, the process data was then processed using IS.

### Step 4 Split of Data Set

Optionally, IOCDC method was used to split the data set into the training set and the test set according to the ratio of 7:3. The number of samples in the training set was 100, and the number of samples in the test set was 44.

### Step 5 Establishment of a Prediction Model

The prediction model was configured to predict the concentration of the bacterium and the C source at the moment t+2 based on the process data at the moment t. The modelling sub-steps were as follows.

### S100 Construction of Multi-Scale Filters (MCFF)

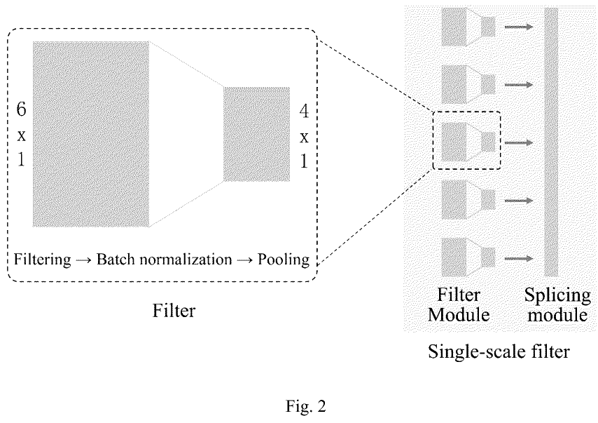

Optionally, the MCFFs were designed with 3 filter module sizes of [2, 1], [3, 1], [4, 1]. Optionally, as shown in FIG. 2, 5 filter modules were provided in each MCFF. The single-channel process data was mapped into 5-channel high-dimensional features, 5 different feature maps were obtained, and then spliced and processed. Preferably, in each filter module, the input data size was [6×1]; the window sliding operation used ‘0’ for padding; a step size was 1; batch normalization operation and ReLU activation function were used; maximum pooling (pooling kernel: [3, 1]) was used for data dimensionality reduction; each filter module output a feature map of [4×1]; and each MCFF output a feature map of [4×1×5].

### S200 Feature Collaborative Hybrid

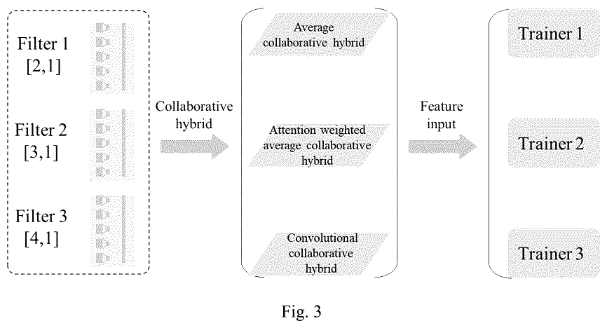

Optionally, as shown in FIG. 3, three collaborative hybrid strategies, for example average collaborative hybrid, attention weighted average collaborative hybrid, and convolutional collaborative hybrid, were used in this study to achieve collaborative hybrid of feature map data for each MCFF.

The mentioned average collaborative hybrid meant that the features at the three scales were averaged to take the value, and the calculation formula was expressed as:

$$X\_average \_hybrid = \sum {X}_{[ i , 1 ]} / 3 ; \qquad ( 3 )$$

where X_{[i,1]} was the feature map vector at different scales, i=2, 3, 4.

The mentioned attention weighted average collaborative hybrid meant that the average pooling was first used to extract the attention values of the output feature maps at each scale, then the attention factors were added, and finally activated using the activation function; and the calculation formula was expressed as:

$$Value\_attention ( i ) = AveragePooling ( X [ i , 1 ] ) , \qquad ( 4 )$$

$$i = 2 , 3 , 4$$

$$Sigmoid ( x ) = 1 / ( 1 + exp ( - x ) ) \qquad ( 5 )$$

$$Attention ( i ) = Sigmoid ( α * Value\_attention ( i ) ) \qquad ( 6 )$$

$$X\_attention \_average \_hybrid = ( \sum Attention ( i ) * {X}_{[ i , 1 ]} ) / 3. \qquad ( 7 )$$

In above formulas, X[i, 1] was the feature map vector at different scales; α was the attention factor with the value of 0.8, and i=2, 3, 4.

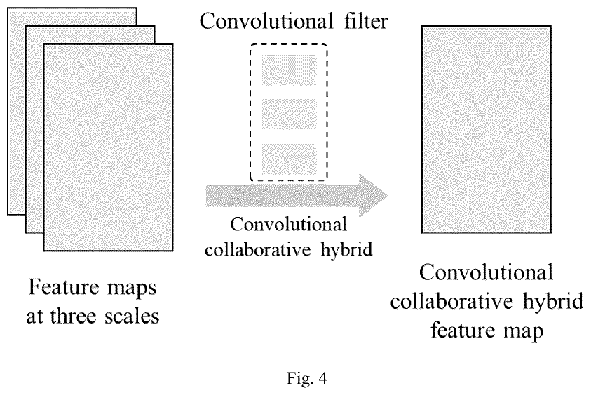

The mentioned convolutional collaborative hybrid referred to the mapping of features at three scales into a new space by a convolutional method, calculated as shown in FIG. 4. Preferably, the convolutional filter contained three convolutional kernels, size of each convolutional kernel was [3, 1], a step size was 1, and ‘0’ was used for padding.

### S300 Put-into Trainer

Optionally, the classical linear algorithm, for example partial least squares (PLS) regression, was used as a trainer for extracting features. The whole architecture was based on gradient descent using backpropagation to adjust the weights of the network. The evaluation parameters of the model were as follows:

$$RMSE = \sqrt{\frac{1}{n} {\sum }_{i = 1}^{n} {( {y}_{i} - {y}_{i ( pre )} )}^{2}} ; \qquad ( 8 )$$

$$and$$

$${R}^{2} = 1 - \frac{{\sum }_{i = 1}^{n} {( {y}_{1} - {y}_{1 ( pre )} )}^{2}}{{\sum }_{i = 1}^{n} {( {y}_{i} - \overline{y} )}^{2}} . \qquad ( 9 )$$

### Step 6 Evolutionary Algorithm Used as Preferred Optimization Algorithm

The evolutionary algorithm parameters were set. Optionally, the number of individuals was 20, the maximum number of iterations was 100, the range of individual gene changes was [−5, 5], and the length of individuals was 16.

### Step 7 Individual Coding

Preferably, the individuals were coded using binary coding. Since the adjustable parameters were limited to dissolved oxygen and pH, each variable was coded using a binary number of 8 bits, for a total of 16 bits, in order to project the data from the low-dimensional space to the high-dimensional space, and to increase the likelihood of obtaining a better solution.

### Step 8 Fitness Function

Taking the change rates of the C source concentration and the bacteria concentration as the individual fitness, combinations of pH and DO with the maximum rate of change were solved. Optionally, the ranges of change of pH and DO were limited.

### Step 9 Establishment of a Simulation System for Optimization Control of Fermentation Process of Kombucha

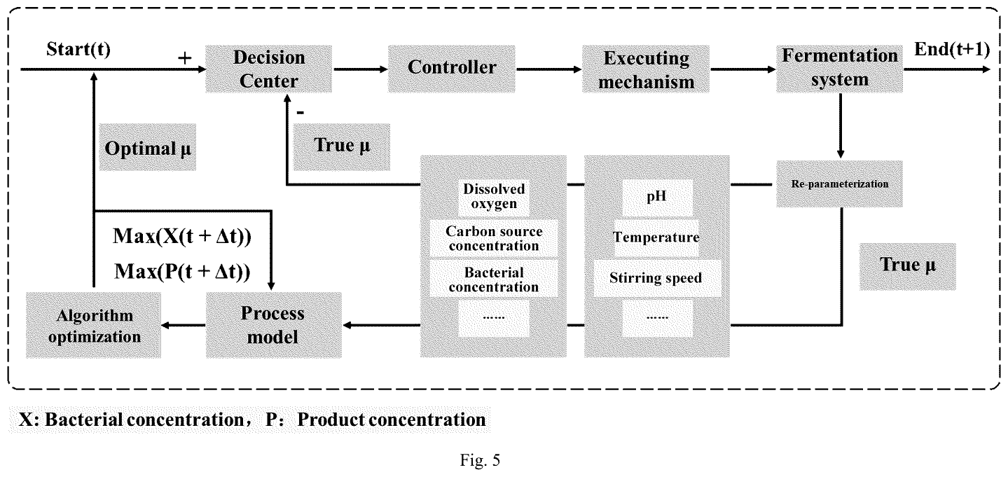

As shown in FIG. 5, an optimal combination of pH and DO was solved based on the current process data, the process model, and the optimization algorithm at the fermentation moment t. According to the different regulation objectives, three regulation modes were designed, namely, the optimization control of bacterium concentration, the optimization control of C source consumption, and the segmented optimization control of the bacterium concentration in a former stage and the C source consumption in a later stage. The simulation system was developed using Matlab 2014b.

The above steps were the simulation application of the method of the disclosure in the fermentation process of kombucha. The model and the optimization control system were validated using the fifth batch of process data. The validation results as follows showed that the model was highly accurate, and the optimization control was effective.

As shown in Table 1, during the establishment of the C-source prediction model, after feature extraction, the performance of the single-scale model with scale 3-PLS was higher than that of the PLS model. The PLS model with convolutional collaborative hybrid multiscale features achieved the best performance, with an R_{P}² of 0.9677 and an RMSEP of 0.5538 mg/mL. The results indicated that the multiscale feature collaborative hybrid strategy could improve the performance of PLS model; and compared with the PLS model, the convolutional collaborative hybrid-PLS model improved the accuracy by 42.3%, which could be used for prediction and monitoring of C source during fermentation.

TABLE 1
Results of C source prediction model C source unit: mg/mL

| | Number of | | | | |
|---|---|---|---|---|---|
| Modelling | principal | Training set | | Test set | |
| method | Components | R_{C}² | RMSEC | R_{P}² | RMSEP |
| PLS | 6 | 0.9171 | 0.9144 | 0.9028 | 0.9604 |
| Scale 1-PLS | 10 | 0.9189 | 0.9188 | 0.8745 | 1.0912 |
| Scale 2-PLS | 10 | 0.9546 | 0.6880 | 0.9462 | 0.7147 |
| Scale 3-PLS | 10 | 0.9711 | 0.5484 | 0.9616 | 0.6035 |
| Average | 10 | 0.9236 | 0.8922 | 0.8979 | 0.9843 |
| collaborative | | | | | |
| hybrid-PLS | | | | | |
| Attention | 10 | 0.9229 | 0.8963 | 0.8972 | 0.9879 |
| weighted average | | | | | |
| collaborative | | | | | |
| hybrid-PLS | | | | | |
| Convolutional | 10 | 0.9720 | 0.5399 | 0.9677 | 0.5538 |
| collaborative | | | | | |
| hybrid-PLS | | | | | |

As shown in Table 2, during the establishment of the bacterial concentration prediction model, after feature extraction, the performance of the single-scale models was all higher than that of the PLS model; the PLS model with convolutional collaborative hybrid multiscale features achieved the best performance, with an R_{P}² of 0.9759 and an RMSEP of 0.0558 Au. The results showed that the multiscale feature collaborative hybrid strategy can improve the performance of the PLS model; and the convolutional collaborative hybrid-PLS model improved the accuracy by 63.7% compared with PLS model, which could be used for prediction and monitoring of bacterial concentration during fermentation.

TABLE 2
Results of bacterial concentration prediction model Bacterial
concentration unit: Au

| Modelling | Principal | Training set | | Test set | |
|---|---|---|---|---|---|
| method | Component | R_{C}² | RMSEC | R_{P}² | RMSEP |
| PLS | 6 | 0.9092 | 0.1275 | 0.8172 | 0.1536 |
| Scale 1-PLS | 10 | 0.9534 | 0.0913 | 0.9279 | 0.0965 |
| Scale 2-PLS | 10 | 0.9689 | 0.0746 | 0.0837 | 0.9458 |
| Scale 3-PLS | 10 | 0.9764 | 0.0650 | 0.9717 | 0.0606 |
| Average | 10 | 0.9628 | 0.0816 | 0.9593 | 0.0725 |
| collaborative | | | | | |
| hybrid-PLS | | | | | |
| Attention | 10 | 0.9629 | 0.0815 | 0.9588 | 0.0729 |
| weighted average | | | | | |
| collaborative | | | | | |
| hybrid-PLS | | | | | |
| Convolutional | 10 | 0.9858 | 0.0504 | 0.9759 | 0.0558 |
| collaborative | | | | | |
| hybrid-PLS | | | | | |

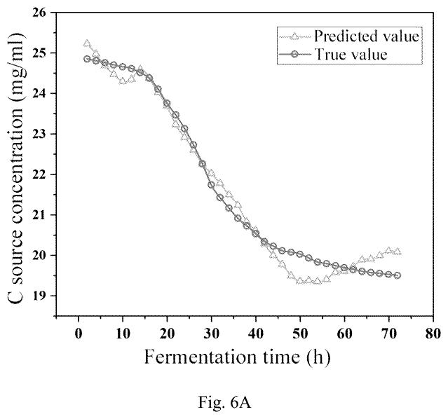

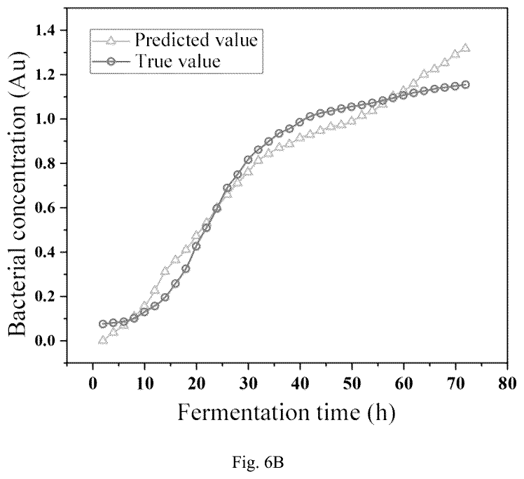

As shown in FIGS. 6A and 6B, the prediction model was tested using the fifth batch of process data, and the prediction results of C source (FIG. 6A) and bacterial concentration (FIG. 6B) were basically in line with the real change trend, and the validation results were good, which indicated that the established prediction model was stable and reliable.

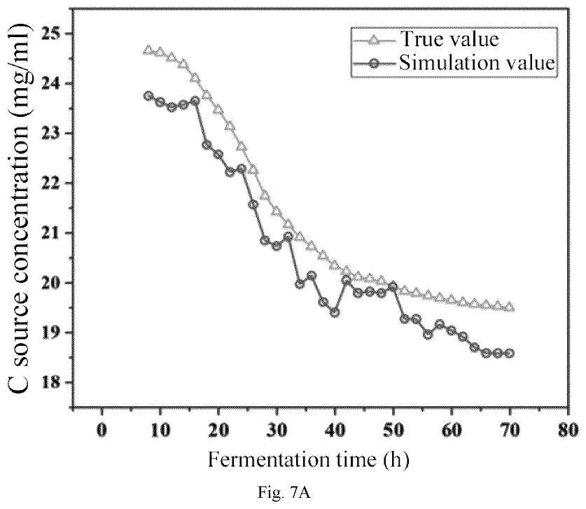

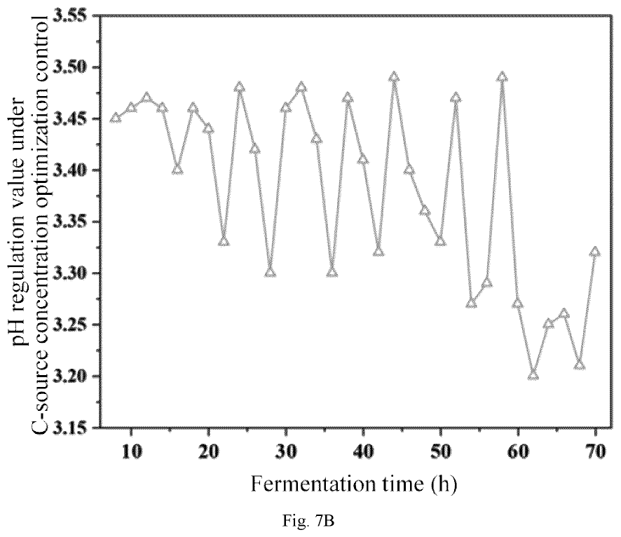

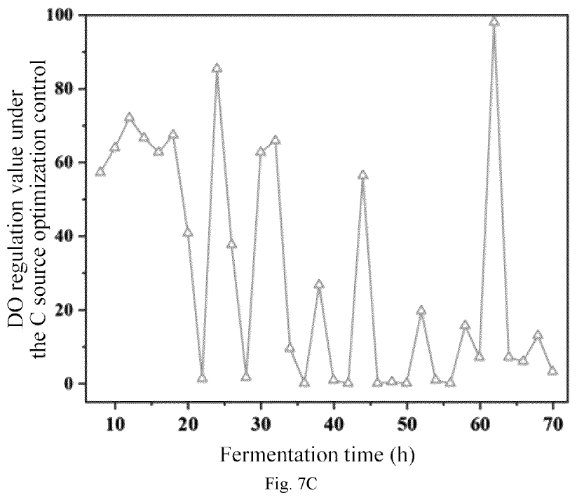

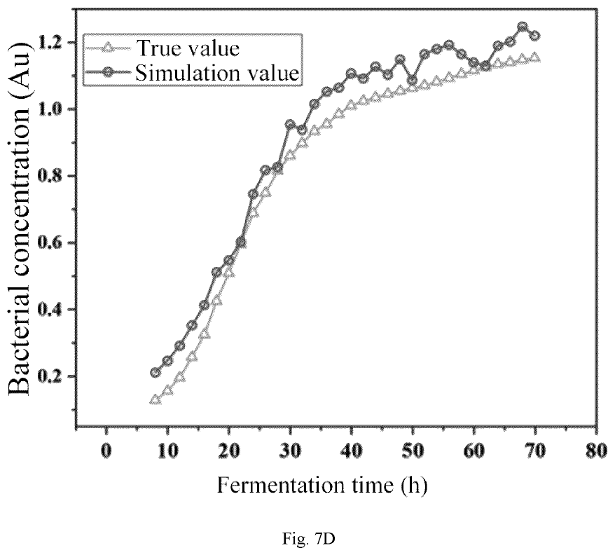

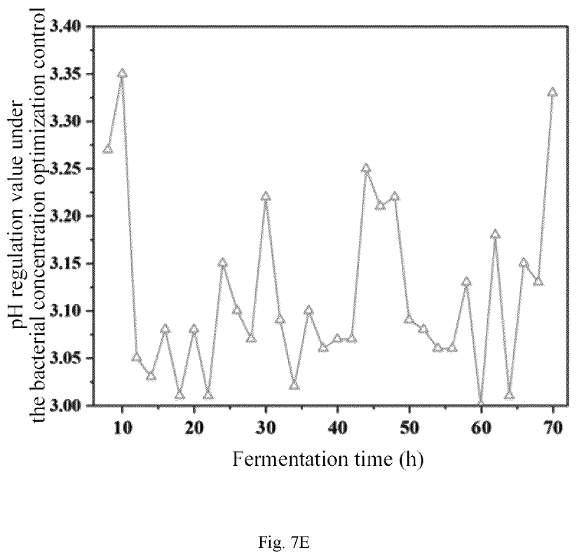

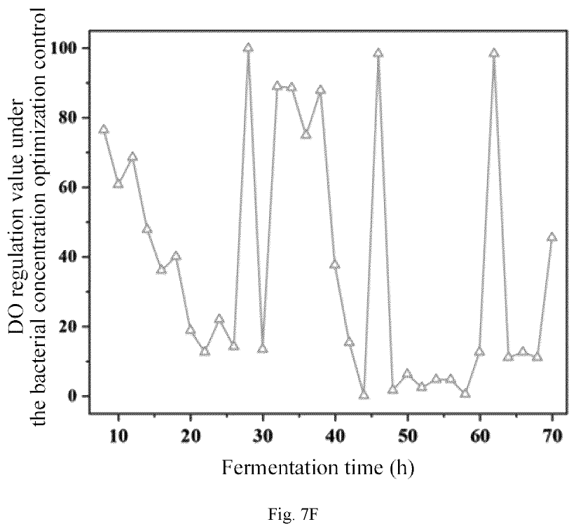

As shown in FIGS. 7A-7F, when the optimization control of C source was performed, the optimization time was 6 h-70 h, and a better parameter combination of pH (FIG. 7B) and DO (FIG. 7C) was solved at each moment, which led to faster consumption of C source (FIG. 7A), which indicated that the consumption rate of C source could be increased, and more energy could be supplied. When the optimization control of bacterial concentration was performed, the optimization time was 6 h-70 h, and the better parameter combination of pH (FIG. 7E) and DO (FIG. 7F) was solved at each moment to obtain a larger concentration of bacterial concentration (FIG. 7D), which indicated that a larger number of cells could be obtained, thereby laying the foundation for obtaining more products.

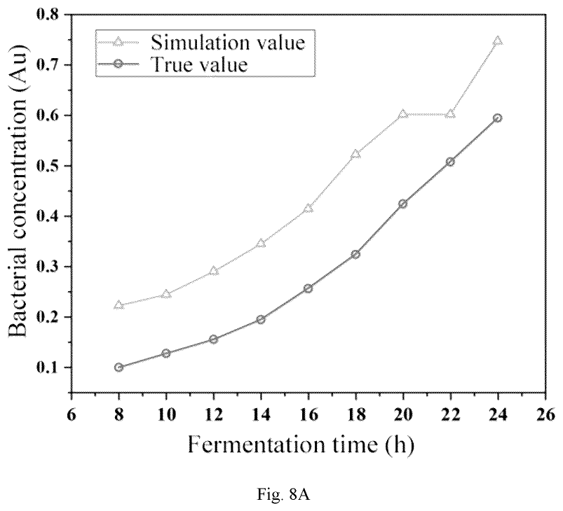

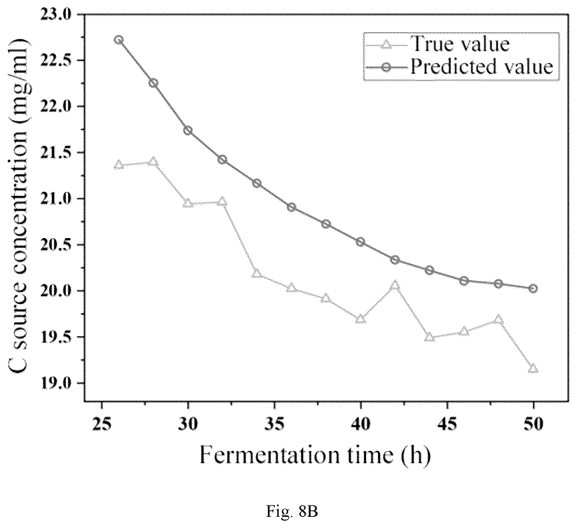

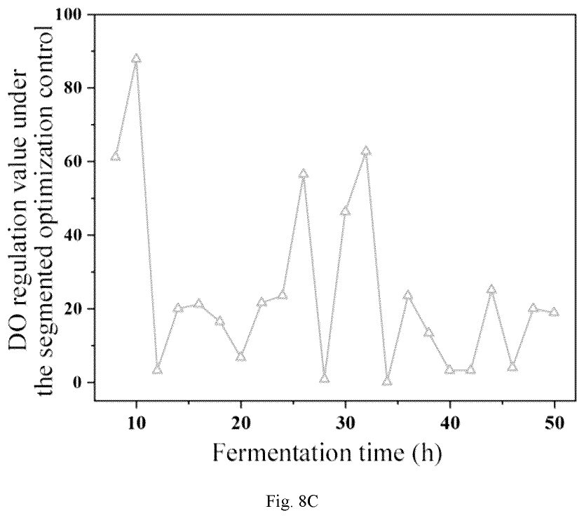

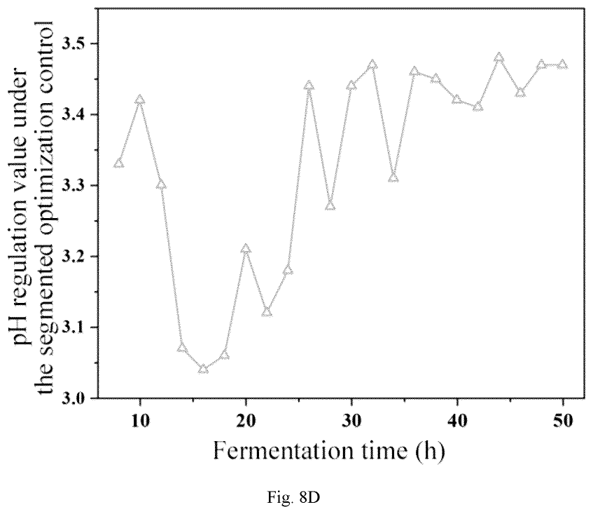

However, the regulation of a single indicator could not meet the needs of the whole fermentation process, so the segmented fermentation optimization control was designed. The bacterial concentration was regulated in 8 h-24 h, and the C source was regulated in 26 h-50 h. As shown in FIGS. 8A-8D, the bacterial concentration was increased (FIG. 8A), the C source consumption was improved (FIG. 8B), and the best combination of DO (FIG. 8C) and pH (FIG. 8D) required to be regulated in the segmented optimization control process were solved.

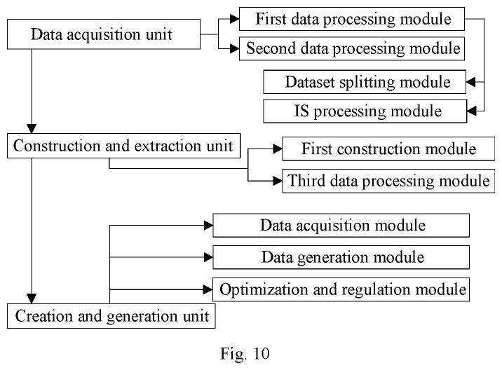

In order to achieve the above objects, the disclosure also provides a data optimization system applicable to the food fermentation process, as shown in FIG. 10. The system is applied to perform the described data optimization method. The data optimization system includes a data acquisition unit, a construction and extraction unit, and a creation and generation unit.

The data acquisition unit is configured to acquire process data of the food fermentation process. The process data includes the environmental parameter data and the fermentation system parameter data.

The construction and extraction unit is configured to construct the MCFF, extract feature data corresponding to the process data in real time based on the MCFF, and process the feature data.

The creation and generation unit is configured to create the data prediction model corresponding to the feature data through the corresponding machine learning method, and generate predicted optimization control data corresponding to the food fermentation process in real time based on the data prediction model in combination with an optimization algorithm. The machine learning method includes the linear method and the non-linear method. The optimization algorithm is the swarm intelligence algorithm or the meta-heuristic algorithm.

The data acquisition unit includes a first data processing module and a second data processing module. The first data processing module is configured for constructing the process data set corresponding to the process data and processing the process data in the process data set by IS. The second data processing module is configured for coupling the process data at the current moment with target value data at the next moment.

The construction and extraction unit includes a first construction module and a third data processing module.

The first construction module is configured for adjusting a size of each filter module to construct the MCFF in real time. The MCFF includes at least one filter module and at least one splicing module. The number of the at least one filter module is adjustable. Each filter module includes a filtering sub-module, a batch normalization sub-module, an activation sub-module, and a pooling sub-module. The filter modules have the same size. The splicing module is configured for integrating the output of the filter module.

The third data processing module is configured for processing the feature data in real time by using the corresponding one filter module. The filtering sub-module extracts features of input parameters. The batch normalization sub-module processes filtered data to reduce the overfitting probability. The activation sub-module performs the non-linear mapping of normalization data. The pooling sub-module reduces the dimensionality of mapped data.

The creation and generation unit includes a data acquisition module, a data generation module, and an optimization and regulation module.

The data acquisition module is configured for generating and acquiring indicator regulation data corresponding to the food fermentation process in real time.

The data generation module is configured for establishing the indicator regulation process prediction model corresponding to the indicator regulation data, and generating optimization algorithm parameter data corresponding to the indicator regulation process prediction model.

The optimization and regulation module is configured for encoding and processing the indicator regulation data, and optimizing and regulating the food fermentation process in real time according to the optimization algorithm parameter data in combination with sampling frequency data or sampling interval data of the process data. The indicator regulation data is encoded by the floating-point encoding and the binary encoding.

The first data processing module further includes a dataset splitting module configured for splitting the process data set into the training set and the test set in accordance with the preset ratio through the IOCDC method. The preset ratio of the training set and the test set is 7:3.

The first data processing module further includes an IS processing module configured for processing the process data in real time in conjunction with max-min normalization.

In the system embodiment of the disclosure, the specific detailed steps involved in the data optimization method applicable to the food fermentation process have been set out above. In other words, the functional modules in the system are used to perform the steps or sub-steps in the data optimization method, which are not repeated here.

The data optimization method in the disclosure acquires process data of the food fermentation process. The process data includes environmental parameter data and fermentation system parameter data. The method constructs the MCFF, extracts feature data corresponding to the process data in real time based on the MCFF, and collaboratively processes the feature data. The method creates the data prediction model corresponding to the feature data through the machine learning method, and based on the data prediction model in combination with the optimization algorithm, generates predicted optimization control data corresponding to the food fermentation process in real time. The machine learning method includes the linear method and the non-linear method. The optimization algorithm is the swarm intelligence algorithm or the meta-heuristic algorithm. The data optimization method and the data optimization system corresponding to the method realize the high-level transformation of the features and complete process data deep mining to establish the reliable process model. Based on the process model, the optimization control system is built in combination with the optimization algorithm, which solves the problem of mutual coupling of process parameters.

In view of the challenges of modelling and optimization control in the food fermentation process, and considering the high-level feature extraction of process data, this application designs a data-driven and multi-scale feature extraction-based approach for modelling and optimization control in the food fermentation process. The MCFF feature extraction tool is designed to achieve multi-scale feature extraction of process data through filtering operations, and collaboratively mix feature data at different scales, thereby achieving high-level transformation of features and completing the deep mining of process data to establish a reliable process model. The optimization control system is built based on the process model and the optimization algorithm, thereby solving the difficult problem of the process parameters coupling with each other. The accuracy of the prediction model and the effectiveness of the optimization control method are verified by taking the fermentation process of kombucha as an example.

The disclosure provides the data-driven and multi-scale cross-correlation feature extraction-based method for modelling and optimization control in the food fermentation process, and relates to microbial fermentation engineering technology. The specific steps are as follows. Firstly, feature extraction of input parameters in the fermentation process of kombucha is carried out using the constructed MCFF. Secondly, the extracted features are collaboratively mixed (performed with collaborative hybrid) and downscaled. Then, the collaborative hybrid features are used as inputs to the PLS to train the process prediction model. Subsequently, predicted values of the process prediction model are used as the basis for the optimization and control. Finally, the evolutionary algorithm is used to solve the optimal combination of the fermentation parameters. While building the C-source prediction model, the PLS model with convolutional collaborative hybrid multiscale features achieved the best performance, with the R_{P}² of 0.9677 and the RMSEP of 0.5538 mg/mL, which improved the accuracy by 42.3% compared with the PLS model. While building the bacterial concentration prediction model, the PLS model with convolutional collaborative hybrid multi-scale features achieved the best performance with the R_{P}² of 0.9759 and the RMSEP of 0.0558 Au, which improved the accuracy by 63.7% compared to the PLS model. The results showed that the prediction models of C source and bacterial concentration were stable and reliable, and could be used in the optimization control of fermentation process. According to the different control objectives, three control models were designed in the fermentation process of kombucha, namely, the optimization control of bacterial concentration, the optimization control of C-source consumption, and the segmented optimization control including bacterial concentration in the first stage and C-source consumption in the second stage. All the three control schemes achieved satisfactory results. The simulation results demonstrated that the method could satisfy the modelling and optimization control in the food fermentation process.

A data optimization system provided includes a processor and a memory. The above-described a data acquisition unit, a construction and extraction unit, and a creation and generation unit are stored in the memory as program units. The processor executes the above-described program units stored in the memory to realize corresponding functions.

This disclosure provides a storage medium having the program stored thereon which implements the data optimization method when executed by the processor.

This disclosure provides a processor which is used to run a program. The program executes the data optimization method when run.

The data optimization device includes at least one processor, at least one memory connected to the processor, and a bus. The communication between the processor and the memory is completed via the bus. The processor is used to call program instructions in the memory to execute the data optimization method. The data optimization device herein may be a server, a PC, a PAD, or a cell phone.

This disclosure provides a computer program product configured to perform the data optimization method when executed on the data processing device.

The disclosure is described in conjunction with flowcharts and/or block diagrams of methods, devices (systems), and computer program products according to embodiments of the present disclosure. Each of the processes and/or boxes in the flowchart and/or block diagram, and the combination of processes and/or boxes in the flowchart and/or block diagram, may be implemented by computer program instructions. These computer program instructions may be configured in the processor of a general-purpose computer, a special-purpose computer, an embedded processor, or other programmable data-processing devices to produce the devices such that the instructions executed by the processor of the computer or other programmable data-processing devices produce a device for carrying out the functions specified in the one process or multiple processes of the flowchart and/or one box or multiple boxes of the box diagram.

In an embodiment, the device includes one or more processors (CPUs), the memory, and the bus. The device may also include input/output interfaces and network interfaces.

The memory may include volatile memory in the computer-readable medium, random-access memory (RAM) and/or non-volatile memory, such as read-only memory (ROM) or flash memory (flash RAM). The memory includes at least one memory chip. The memory is an example of a computer readable medium.

Computer-readable medium includes volatile and non-volatile, removable and non-removable media, which may be implemented by any method or technique for information storage. The information may be computer-readable instructions, data structures, or program modules. In some embodiments, the storage media for computers includes, but are not limited to, phase-change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technologies, compact disc read-only memory (CD-ROM), Digital Versatile Disc (DVD) or other optical storage, magnetic cartridge tape, magnetic tape disk storage or other magnetic storage device or other non-transfer medium that can be used to store information that can be accessed by the computing device. In this disclosure, the computer-readable media does not include transitory computer-readable media, such as modulated data signals and carriers.

Described above are merely some embodiments of the disclosure, which are not intended to limit the disclosure. It should be understood that any modifications, and replacements made by those skilled in the art without departing from the spirit of the disclosure should fall within the scope of the disclosure defined by the appended claims.

What is claimed is:

1. A data optimization method of a food fermentation process, comprising:
(a) acquiring process data of the food fermentation process; wherein the process data comprises environmental parameter data and fermentation system parameter data;
(b) constructing a multi-scale cross-correlation feature filter (MCFF); extracting feature data corresponding to the process data in real time based on the MCFF; and processing the feature data by collaborative hybrid; and
(c) creating a data prediction model corresponding to the feature data through a machine learning method; and based on the data prediction model in combination with optimization algorithm, generating predicted optimization control data an corresponding to the food fermentation process in real time; wherein the machine learning method comprises a linear method and a non-linear method; and the optimization algorithm is a swarm intelligence algorithm or a meta-heuristic algorithm.

2. The data optimization method of claim 1, wherein the step (a) further comprises:
(a1) constructing a process data set based on the process data, and processing the process data in the process data set by interval scaling (IS); and
(a2) coupling the process data at a current moment with target value data at a next moment.

3. The data optimization method of claim 2, wherein the step (a1) further comprises:
splitting the process data set into a training set and a test set according to a preset ratio through an input-output cooperative distance classification (IOCDC) method.

4. The data optimization method of claim 2, wherein an IS function is expressed as follows:
$${X}_{i} = ( {X}_{oi} - {X}_{oi , min} ) / ( {X}_{oi , max} - {X}_{oi , min} ) ; \qquad ( 1 )$$
wherein Xₒᵢ is real-time feature data before the IS; and X_{oi,max} and X_{oi,min} represent maximum value and minimum value of individual dimensions, respectively.

5. The data optimization method of claim 1, wherein the step (b) further comprises:
adjusting a size of each of at least one filter module to construct the MCFF in real time, wherein the MCFF comprises the at least one filter module and at least one splicing module; the number of the at least one filter module is adjustable; each of the at least one filter module comprises a filtering sub-module, a batch normalization sub-module, an activation sub-module, and a pooling sub-module; the at least one filter module has the same size; and the at least one splicing module is configured for integrating an output of the at least one filter module; and
processing the feature data in real time by using a corresponding one of the at least one filter module through steps of:
extracting, by the filtering sub-module, features of an input parameter; processing, by the batch normalization sub-module, extracted features of the input parameter to reduce an overfitting probability; performing, by the activation sub-module, a non-linear mapping of normalization data; and reducing, by the pooling sub-module, a dimensionality of mapped data.

6. The data optimization method of claim 1, wherein the step (c) further comprises:
generating and acquiring indicator regulation data corresponding to the food fermentation process in real time;
establishing an indicator regulation process prediction model corresponding to the indicator regulation data, and generating optimization algorithm parameter data corresponding to the indicator regulation process prediction model; and
encoding the indicator regulation data; and optimizing and regulating the food fermentation process in real time according to the optimization algorithm parameter data in combination with sampling frequency data or sampling interval data of the process data; wherein the indicator regulation data is encoded by floating-point encoding and binary encoding.

7. A data optimization system of a food fermentation process, comprising:
a data acquisition unit;
a construction and extraction unit; and
a creation and generation unit;
wherein the data acquisition unit is configured to acquire process data of the food fermentation process; wherein the process data comprises environmental parameter data and fermentation system parameter data;
the construction and extraction unit is configured to construct a multi-scale cross-correlation feature filter (MCFF), extract feature data corresponding to the process data in real time based on the MCFF, and process the feature data by collaborative hybrid; and
the creation and generation unit is configured to create a data prediction model corresponding to the feature data through a machine learning method, and generate predicted optimization control data corresponding to the food fermentation process in real time based on the data prediction model in combination with an optimization algorithm; wherein the machine learning method comprises a linear method and a non-linear method; and the optimization algorithm is a swarm intelligence algorithm or a meta-heuristic algorithm.

8. The data optimization system of claim 7, wherein the data acquisition unit comprises a first data processing module and a second data processing module; the first data processing module is configured for constructing a process data set based on the process data and processing the process data in the process data set by interval scaling (IS); and the second data processing module is configured for coupling the process data at a current moment with target value data at a next moment;
the construction and extraction unit comprises a first construction module and a third data processing module; the first construction module is configured for adjusting a size of each of at least one filter module to construct the MCFF in real time, wherein the MCFF comprises the at least one filter module and at least one splicing module, and the number of the at least one filter module is adjustable; each of the at least one filter module comprises a filtering sub-module, a batch normalization sub-module, an activation sub-module, and a pooling sub-module; the at least one filter module has the same size; and the at least one splicing module is configured for integrating an output of the at least one filter module;
the third data processing module is configured for processing the feature data in real time by using a corresponding one of the at least one filter module through steps of:
extracting features of an input parameter by the filtering sub-module; processing filtered data to reduce an overfitting probability by the batch normalization sub-module; performing a non-linear mapping of normalization data by the activation sub-module; and reducing a dimensionality of mapped data by the pooling sub-module;
the creation and generation unit comprises a data acquisition module, a data generation module and an optimization and regulation module; the data acquisition module is configured for generating and acquiring indicator regulation data corresponding to the food fermentation process in real time;
the data generation module is configured for establishing an indicator regulation process prediction model corresponding to the indicator regulation data, and generating optimization algorithm parameter data corresponding to the indicator regulation process prediction model; and
the optimization and regulation module is configured for encoding the indicator regulation data; and optimizing and regulating the food fermentation process in real time according to the optimization algorithm parameter data in combination with sampling frequency data or sampling interval data of the process data; wherein the indicator regulation data is encoded by floating-point encoding and binary encoding.

9. The data optimization system of claim 8, wherein the first data processing module comprises a dataset splitting module configured for splitting the process data set into a training set and a test set through an input-output cooperative distance classification (IOCDC) method according to a preset ratio, wherein a cooperative distance calculation formula is expressed as:
$${d}_{xy} ( i , j ) = \frac{{d}_{x} ( i , j )}{{max}_{i , j ∈ ( 1 , z )} ( {d}_{x} ( i , j ) )} + \frac{{d}_{y} ( i , j )}{{max}_{i , j ∈ ( 1 , z )} ( {d}_{y} ( i , j ) )} ; \qquad ( 1 )$$
wherein i, j∈[1, z], z is the number of samples; dₓ(i, j) is an input-based inter-sample distance; and d_{y}(i, j) is an output-based inter-sample distance.

10. The data optimization system of claim 8, wherein the first data processing module comprises an IS processing module configured to process the process data in real time through IS, wherein an IS function is expressed as follows:
$${X}_{i} = ( {X}_{oi} - {X}_{oi , min} ) / ( {X}_{oi , max} - {X}_{oi , min} ) . \qquad ( 2 )$$
wherein Xₒᵢ is real-time feature data before the IS; and X_{oi,max} and X_{oi,min} represent maximum value and minimum value of individual calculation dimensions, respectively.
